(19) United States
(12) Patent Application Publication
(10) Pub. No.: US 2017/0368021 A1
(43) Pub. Date: Dec. 28, 2017

(54) MEDICATED PRODUCE

(71) Applicants: Walker Atkinson, Denver, CO (US); Megan Costello, Breck, CO (US)

(72) Inventors: Walker Atkinson, Denver, CO (US); Megan Costello, Breck, CO (US)

(21) Appl. No.: 15/632,574

(22) Filed: Jun. 26, 2017

Related U.S. Application Data
(60) Provisional application No. 62/354,811, filed on Jun. 27, 2016.

(51) Int. Cl. A61K 31/352; A23L 33/105; A23L 19/00; A61K 31/05

(52) U.S. Cl. CPC A61K 31/352; A61K 31/05; A23L 33/105; A23L 19/00; A23V 2002/00

(57) ABSTRACT

Methods and products of processes result in novel medicated food items including produce items. Cannabinoids from marijuana plants, including THC, CBD and CBC are infused with fresh fruits and vegetables, resulting in an innovative approach combining cannabinoid ingestion with healthy eating.

### REFERENCE TO RELATED APPLICATIONS

This application claims priority to U.S. Provisional Patent Application Ser. No. 62/354,811, filed Jun. 27, 2016, the entire content of which is incorporated herein by reference.

### FIELD OF THE INVENTION

This invention relates generally to food additives and, in particular, to methods and products of processes used to medicate produce and other food items.

### BACKGROUND OF THE INVENTION

There are numerous compounds, known as cannabinoids, found in the marijuana or cannabis plant. THC (tetrahydrocannabinol), for example, is the cannabinoid responsible for many of the psychoactive effects experienced by cannabis users. The majority of marijuana plant strains include THC in a range from 12-21%, and higher in some cases. Though psychoactive, THC also provides numerous therapeutic effects, including mild to moderate pain relief, relaxation and anti-depressant effects.

While many people have heard of THC, cannabis also includes other cannabinoids with enhanced therapeutic effects in conjunction with fewer—if any—psychoactive effects. Most people have never heard of these other cannabinoids.

Cannabidiol (CBD), for example, occurs in many strains, but typically at low levels of 1% or less. In rare cases, CBD can be the dominant cannabinoid. Research has determined that CBD can provide relief from chronic pain due to muscle spasticity, convulsions and inflammation. As such, CBD can provide relief for patients with MS, Fibromyalgia and Epilepsy. Some researchers believe CBD also provides effective relief from anxiety-related disorders and may inhibit cancer cell growth when injected into tumors in combination with THC.

Cannabichromene (CBC) is a rare, non-psychoactive cannabinoid, also found at low levels of less than 1%. Research has shown, however, that CBC has profound anti-depressant effects, ten times those of CBD. CBC has also been shown to improve the pain-relieving effects of THC, with sedative effects promoting relaxation.

Other cannabinoids include cannabigerol (CBG), a non-psychoactive cannabinoid shown to provide pain relief, inflammation reduction, and intraocular pressure associated with glaucoma. CBG may also have antibiotic properties that inhibit platelet aggregation, which slows the rate of blood clotting. Cannabinol (CBN), an oxidized form of THC, has some psychoactive properties but few therapeutic effects.

Currently in the United States, the marijuana/cannabis business is booming. This multi-billion dollar industry has expanded from just growing and selling the marijuana plant and buds to extracting the oils and other elements of the plant including THC, CBD and CBC. These compounds are combined with other substances to create edibles, vapors and oils.

Medical marijuana is now a very common remedy for treating many medical issues, including those identified above. While suffering from many of these debilitating diagnoses, maintaining a healthy eating lifestyle is recommended. In particular, fresh produce in the form of fruits and vegetables are a recommended way to obtain necessary nutrients, vitamins and minerals needed for optimal health.

The needs remains, however, for an innovative approach to combine cannabinoid ingestion with healthy eating.

### SUMMARY OF THE INVENTION

In view of the foregoing disadvantages inherent in the known art, the present invention provides for methods and products of processes resulting in novel medicated food items including produce items. In the preferred embodiments, cannabinoids from marijuana plants, including THC, CBD and CBC infused with fresh fruits and vegetables. Where the food item is a citrus fruit having edible sections divided by separating walls, the method may include the step of determining the positions of the separating walls to ensure that the cannabinoid is injected into one or more of the edible sections.

Whereas injection with a syringe is the preferred method of introducing the cannabinoid, other infusion methods are possible including soaking and genetic modification.

### BRIEF DESCRIPTION OF THE DRAWINGS

FIG. 1 is a simplified diagram illustrating a preferred embodiment of the invention.

### DETAILED DESCRIPTION OF THE PREFERRED EMBODIMENTS

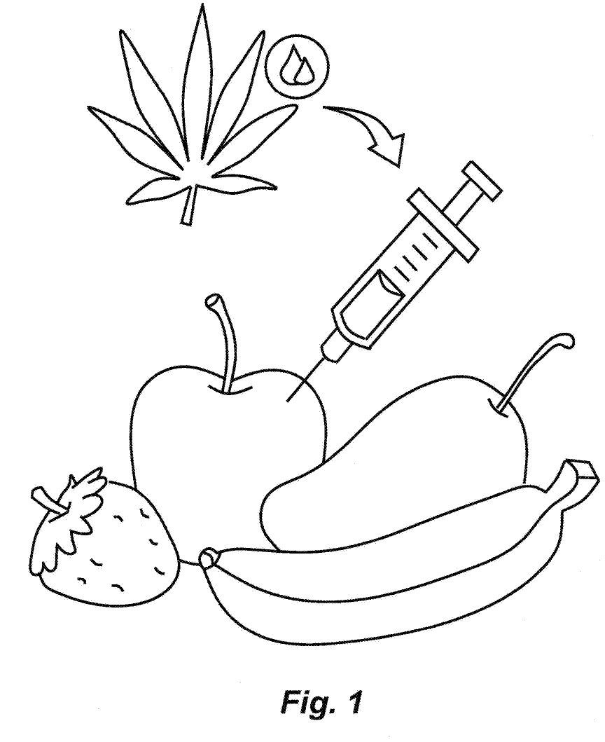

FIG. 1 is a simplified diagram illustrating a process according to the invention. As an initial step, a desired cannabinoid is extracted from the leaves/buds of the cannabis plant. The cannabinoid, typically in the form of a flowable oil, is loaded into a syringe, which is then used to inject the cannabinoid into an item of produce or other appropriate food item.

Processes for extracting cannabinoids are known in the art, and this invention is not limited in terms of the extraction method used. As one example, CBD and other cannabinoids may be extracted using high-proof food-safe alcohol. The plant material mixed with alcohol and stirred or agitated to expel the resin.

The liquid is then filtered with a sieve and collected as an initial raw extraction. The stirring and filtering process can be repeated with a new batch of solvent to extract as much compounds as possible from the plant matter.

The strained liquid is then strained into the double boiler and heated to evaporate the alcohol until a flowable liquid or oil is produced. The flowable liquid or oil may then be stored or loaded directly into syringes for use in accordance with the invention.

For more accurate separation of oils into specific cannabinoids, Flashpoints may be used according to the following table:

| Cannabinoid | Flashpoint |
|---|---|
| tetrahydrocannabivarin (THCV) | 137.6 C. (279.68 F.) |
| delta-8-tetrahydrocannabinol | 144.5 C. (292.10 F.) |
| (delta-8-THC) | |
| delta-9-tetrahydrocannabinol | 149.3 C. (300.74 F.) |
| (THC) | |
| cannabichromene (CBC) | 174.2 C. (345.56 F.) |
| cannabidiol (CBD) | 206.3 C. (403.34 F.) |
| cannabigerol (CBG) | 207.2 C. (404.96 F.) |
| cannabinol (CBN) | 212.7 C. (414.86 F.) |

These flashpoints would then be used in conjunction with a temperature-controlled vaporizer with collection bags and high-grain alcohol. Using such processes, cannabinoids may be collected individually or combined with unwanted cannabinoids being discarded.

For example, to collect CBD alone, the vaporizer would be set just beyond 175 C or 346 F, or just above the flashpoint for CBC, while still remaining safely below that of the CBD to be collected. Pre-CBD content may be ‘purged’ at roughly 390-400 F. This process may be used for any of the items listed in the table based upon the flashpoints given.

Once the desired cannabinoid or mixture of cannabinoids is made available by whatever method, the material is injected into a food article, preferably a fresh fruit or vegetable. The fresh produce may be in many forms, from all stages of growth, from seeds and seedlings to ripe/mature produce ready for harvest using the appropriate syringe or injection method. The following example describes one embodiment of the invention:

### Example

### 300 mg CBD Oil

Fruits (i.e., oranges, banana, watermelon)
Vegetables (i.e., carrots, cucumber, zucchini)
In the case of citrus fruits, look for humps for slices or use X-ray to determine where slices are to ensure that the injection is made into edible fruit itself and not the partitions between the fruit slices.

### Needles/Syringes:

Syringe size: 3.4″×0.9″×0.6″/8.7×2.4×1.5 cm, Capacity: 3 ml.
Blunt tip needles length (stainless steel part): 14Ga-1.5″/38 mm, 18Ga-0.5″/13 mm, 20Ga-1.5″/38 m, 25Ga-0.5″/13 mm.
Injections of 10, 50 and 100 mg were tested for citrus fruit, bananas, cucumber/zucchini and watermelon.

Note that while injection with a syringe is the preferred method of introducing the cannabinoid, other infusion methods are possible including soaking and genetic modification involving the mutation, insertion, or deletion of fruit/vegetable plant genes. Cannabinoid genetic material may be introduced by attaching the genes to a virus; physically inserting the genetic material into the nucleus of the intended host with a very small syringe; or via electroporation. The preferred technique would exploit more natural forms of gene transfer, such as the ability of agrobacterium to transfer cannabis related genetic material to target plants.

1. A method of producing a medicated food item, comprising the steps of:
providing a food item;
extracting a cannabinoid from a cannabis plant;
loading the cannabinoid into a needle or syringe; and
injecting the cannabinoid into the food item.

2. The method of claim 1, wherein the food item is a fruit or vegetable.

3. The method of claim 1, wherein:
the food item is a citrus fruit having edible sections divided by separating walls; and
including the step of determining the positions of the separating walls to ensure that the cannabinoid is injected into one or more of the edible sections.

4. The method of claim 1, wherein the cannabinoid is a non-psychoactive cannabinoid.

5. The method of claim 1, wherein the cannabinoid is a tetrahydrocannabinol.

6. The method of claim 1, wherein the cannabinoid is cannabidiol (CBD).

7. The method of claim 1, wherein the cannabinoid is cannabichromene (CBC).

8. A medicated food item produced in accordance with the method of claim 1.

9. A medicated food item produced in accordance with the method of claim 2.

10. A medicated food item produced in accordance with the method of claim 3.

11. A medicated food item produced in accordance with the method of claim 4.

12. A medicated food item produced in accordance with the method of claim 5.

13. A medicated food item produced in accordance with the method of claim 6.

14. A medicated food item produced in accordance with the method of claim 7.

15. A medicated food item produced in accordance with the method of claim 8.
